(19) United States
(12) Patent Application Publication
(10) Pub. No.: US 2024/0132834 A1
(43) Pub. Date: Apr. 25, 2024

(54) RECOMBINANT MICROORGANISM INCLUDING GENETIC MODIFICATION THAT INCREASES EXPRESSION OF IRON STORAGE PROTEIN WITH HEME STRUCTURE, AND METHOD OF REDUCING CONCENTRATION OF NITROGEN OXIDE IN SAMPLE USING THE SAME

(71) Applicant: Samsung Electronics Co., Ltd., Suwon-si (KR)

(72) Inventors: Yu Kyung Jung, Suwon-si (KR); Jae-Young Kim, Suwon-si (KR); Seung Hoon Song, Suwon-si (KR); Woo Yong Shim, Suwon-si (KR)

(21) Appl. No.: 18/301,389

(22) Filed: Apr. 16, 2023

(30) Foreign Application Priority Data
Oct. 20, 2022 (KR) 10-2022-0136850

(51) Int. Cl. C12N 1/20; C07K 14/195

(52) U.S. Cl. CPC C12N 1/205; C07K 14/195; C12R 2001/185

(57) ABSTRACT

Provided are a recombinant microorganisms having a genetic modification that increases the expression of bacterioferritin, a composition comprising the recombinant microorganism for use in reducing a nitrogen oxide concentration in a sample, and a method of reducing a nitrogen oxide concentration in a sample.

### CROSS-REFERENCE TO RELATED APPLICATION

This application is based on and claims priority under 35 U.S.C. § 119, and all of the benefits accruing therefrom, to Korean Patent Application No. 10-2022-0136850, filed on Oct. 21, 2022, in the Korean Intellectual Property Office, the content of which is incorporated by reference herein in its entirety.

### INCORPORATION-BY-REFERENCE OF ELECTRONICALLY FILED MATERIAL

The Instant Application contains a Sequence Listing which has been submitted electronically as an XML file and is hereby incorporated by reference in its entirety. Said XML file, created on Apr. 17, 2023, is named “4.S169718US_Sequence List.XML” and is 28,672 bytes in size.

### BACKGROUND

### 1. Field

Provided are recombinant microorganisms that include a genetic modification that increases the expression of an iron storage protein having a heme structure, a composition including the recombinant microorganisms for use in reducing a nitrogen oxide concentration in a sample, and a method of reducing a nitrogen oxide concentration in a sample.

### 2. Description of the Related Art

Nitrogen oxide (NOx) is one of the air pollutants mainly emitted during the combustion process of fuels and examples of various oxides (also referred to as Nox) include N₂O, NO, N₂O₃, NO₂, N₂O₄, and N₂O₅, among which NO and NO₂ are the main causes of air pollution. N₂O absorbs and stores heat in the atmosphere, along with carbon dioxide (CO₂), methane (CH₄), and Freon gas (e.g. fluorochlorocarbons (CFCs)), thereby causing the greenhouse effect. N₂O is one of the six greenhouse gases subject to regulation by the Kyoto Protocol, and has a global warming potential (GWP) of 310, which means that it has a higher warming effect per unit mass than carbon dioxide and methane. In addition, nitrogen oxides are a leading cause of smog and acid rain. Nitrogen oxide, through chemical reactions in the air, forms second generation ultrafine dust, and increases ground-level ozone concentrations, which adversely affects respiratory health.

Most nitrogen oxide removal processes, including technologies such as selective catalytic reduction (SCR) and selective non-catalytic reduction (SNCR), are chemical reduction methods, and scrubbing and adsorption are mainly being applied. The chemical methods have drawbacks such as the cost of energy and catalysts required in the entire process, and the need for treatment of secondary wastes generated therefrom. In addition, in the case of SCR or SNCR, another greenhouse gas, N₂O, may be generated as a result of incomplete reduction when reducing NO and NO₂. Unlike the chemical methods, a biological process is an environmentally friendly process with advantages such as a relatively simple principle, no use of extreme conditions (including, e.g. high temperature and high pressure), and low generation of secondary wastes or waste water. In the biological process, microorganisms acting as biological catalysts are used instead of chemical catalysts to oxidize or reduce NOx, or to fix NOx as a part of the cells.

However, despite advances, there is still a need for alternative methods of biological denitrification.

### SUMMARY

Denitrification microorganisms reduce nitrogen oxides to N₂ through a dissimilatory reduction process. In previous studies, many denitrification microorganisms such as Pseudomonas putida, Pseudomonas denitrificans, Pseudomonas stutzeri, Paracoccus denitrificans, Klebsiella pneumonia, and the like have been reported.

Provided in an aspect is a recombinant microorganism including a genetic modification that increases the expression of an iron storage protein (ISP) having a heme structure.

Provided in an aspect is a composition for use in reducing a nitrogen oxide concentration in a sample, the composition including the recombinant microorganism including a genetic modification that increases the expression of an iron storage protein (ISP) having a heme structure.

Provided in an aspect is a method of reducing a nitrogen oxide concentration in a sample, including reducing a nitrogen oxide concentration in a sample by contacting a nitrogen oxide-containing sample with the recombinant microorganisms including a genetic modification that increases the expression of an iron storage protein (ISP) having a heme structure.

Additional aspects will be set forth in part in the description which follows and, in part, will be apparent from the description, or may be learned by practice of the presented embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A is a diagram showing amounts of Fe(II)EDTA-NO removed during Fe(II)EDTA-NO reduction reaction by using recombinant E. coli in which ftnA, ftnB, bfr, and dps genes are overexpressed;

FIG. 1B is a diagram comparing colors of reaction solutions of Fe(II)EDTA-NO reduction that uses recombinant E. coli in which ftnA, ftnB, bfr, and dps genes are overexpressed;

FIG. 2A is a diagram showing amounts of Fe(II)EDTA-NO removed during Fe(II)EDTA-NO reduction reaction by using recombinant E. coli in which bfd, bfr, and bfd-bfr genes are respectively deleted;

FIG. 2B is a diagram comparing colors of reaction solutions of Fe(II)EDTA-NO reduction that uses recombinant E. coli in which bfd, bfr, and bfd-bfr genes are respectively deleted; and

FIG. 3 is a diagram showing results of measuring norV promoter activity of a strain overexpressing iron storage protein (ISP) gene under Fe(II)EDTA-NO reduction conditions.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects.

The terminology used herein is for the purpose of describing particular embodiments only and is not intended to be limiting. As used herein, “a,” “an,” “the,” and “at least one” do not denote a limitation of quantity, and are intended to cover both the singular and plural, unless the context clearly indicates otherwise. For example, “an element” has the same meaning as “at least one element,” unless the context clearly indicates otherwise. “Or” means “and/or.” “At least one” is not to be construed as limiting “a” or “an.” As used herein, the term “and/or” includes any and all combinations of one or more of the associated listed items. As used herein, the term “or” and “and/or” includes any and all combinations of one or more of the associated listed items. Expressions such as “at least one of,” when preceding a list of elements, modify the entire list of elements and do not modify the individual elements of the list.

It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±20%, 10% or ±5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

The term “increase in activity” or “increased activity”, as used herein, refers to a detectable increase in activity of a cell, protein, or enzyme. “Increase in activity” or “increased activity” refers to a level of activity of a modified cell, protein, or enzyme (for example, modified by genetically engineering) to have a higher level of activity than that of a comparable cell, protein, or enzyme of the same type, not having a given genetic modification (for example, original or “wild-type” cell, protein, or enzyme). “Activity of a cell” refers to activity of a specific protein or enzyme of a cell. For example, activity of the modified or engineered cell, protein, or enzyme may be about 5% or more, about 10% or more, about 15% or more, about 20% or more, about 30% or more, about 50% or more, about 60% or more, about 70% or more, or about 100% or more, greater than the activity of an unengineered cell, protein, or enzyme of the same type, for example, a wild-type cell, protein, or enzyme. Activity of a particular protein or enzyme in a cell may be about 5% or more, about 10% or more, about 15% or more, about 20% or more, about 30% or more, about 50% or more, about 60% or more, about 70% or more, or about 100% or more, increased than the activity of the same protein or enzyme in a parental cell, for example, an unengineered cell. Cells with increased activity of a protein or enzyme may be identified by using any method known in the art.

Increased activity of an enzyme or polypeptide may be obtained by increased expression or increased specific activity. The increased expression may be due to an introduction of a polynucleotide encoding an enzyme or polypeptide into a cell, or an increase in copy number of the polynucleotide, or due to a mutation in a regulatory region of the polynucleotide. The microorganism into which a gene is introduced may endogenously include the gene or may not include the gene. The gene may be operably linked to a regulatory sequence that enables its expression, such as a promoter, a polyadenylation site, or a combination thereof. Polynucleotides introduced into cells or whose copy number is increased may be endogenous or exogenous. The endogenous gene refers to a gene already present in the genetic material of in the microorganism. The exogenous gene refers to a gene not already present in the genetic material but introduced into a cell, and the introduced gene may be homologous or heterologous to the host cell into which it is introduced. “Heterologous” may mean foreign, which is not native.

The term “increase in expression” or “increased expression”, as used herein, refers to a detectable increase in expression of a protein. “Increase in expression” or “increased expression” refers to protein expression of a cell modified (for example, genetically engineered) to have a higher level of expression than that of a comparable cell of the same type, which is the same as a cell not having a given genetic modification (for example, original or “wild-type” cell). For example, protein expression of the modified or engineered cell may be about 5% or more, about 10% or more, about 15% or more, about 20% or more, about 30% or more, about 50% or more, about 60% or more, about 70% or more, or about 100% or more increased than that of an unmodified cell of the same type, for example, protein expression of a wild-type cell. Cells with increased expression of a protein or enzyme may be identified by using any method known in the art.

The increased expression may be due to introduction of a polynucleotide encoding an enzyme or polypeptide into a cell, or an increase in copy number of the polynucleotide encoding an enzyme or polypeptide, or a mutation in a regulatory region of the polynucleotide encoding an enzyme or polypeptide. The microorganism into which a polynucleotide is introduced may endogenously include the polynucleotide or may not include the polynucleotide. The polynucleotide may be operably linked to a regulatory sequence that enables its expression, such as a promoter, a polyadenylation site, or a combination thereof. Polynucleotides introduced from outside or whose copy number is increased may be endogenous or exogenous. Endogenous polynucleotide refers to a polynucleotide already present in the genetic material of the microorganism. Exogenous polynucleotide refers to a polynucleotide introduced into a cell, and the introduced polynucleotide may be homologous or heterologous to the host cell into which it is introduced. “Heterologous” may mean foreign, which is not native.

A “copy number increase” may be due to an introduction or amplification of the gene, and may also include a case in which a gene that does not exist in an unengineered cell is obtained by genetic engineering. The introduction of the gene may be achieved through a vehicle such as a vector. The introduction of the gene may be a transient introduction, in which the gene is not integrated into the genome, or an introduction which includes insertion of the gene into the genome. The introduction may be accomplished when, for example, a vector having an inserted polynucleotide encoding a desired polypeptide is introduced into the cell, and then, the vector is replicated in the cell or the polynucleotide is integrated into the genome. When several polypeptides form a complex to exhibit one nitrogen oxide reductase activity, the “copy number increase” may be an increase in a copy number of the gene encoding one or more polypeptides constituting the complex.

Introduction of the gene may be performed by known methods such as transformation, transfection, and electroporation. The gene may be introduced via a vehicle or may be introduced as itself. In this specification, a “vehicle” includes a nucleic acid molecule capable of delivering another linked nucleic acid. The term “vehicle”, as used herein, may be used interchangeably with “vector”, “nucleic acid construct”, and “cassette”, in terms of referring to a nucleic acid sequence that mediates an introduction of a specific gene. A vector includes, for example, a plasmid or a virus-derived vector. The term “plasmid” refers to a circular double-stranded DNA loop into which additional DNA may be ligated. Vectors may include, for example, plasmid expression vectors, viral expression vectors, such as replication-defective retroviruses, adenoviruses, and adeno-associated viruses, or combinations thereof. The gene may be operably linked to a constitutive promoter or an inducible promoter.

“Genetic engineering”, as used herein, may be performed by a molecular biological method known in the art.

The term “parent cell” refers to an original cell, for example, a cell of the same type as the engineered cell but without the genetic modification. The “parent cell” may be a cell that does not have a particular genetic modification in regard to the particular genetic modification, but may be considered the same cell in other circumstances. Thus, the parent cell may be a cell used as starting material for producing a genetically engineered microorganism with increased expression of a given protein (for example, a protein having about 75% or more sequence identity with bacterioferritin). The same comparison applies to other genetic modifications.

The term “gene”, as used herein, refers to a nucleic acid fragment encoding the information for expressing a specific protein, and may or may not include a regulatory sequence of a 5′-non-coding sequence and/or a 3′-non-coding sequence, or may be free of a regulatory sequence.

In the present specification, “sequence identity” of nucleic acids or polypeptides refers to a degree of identicality of nucleotides (bases) of polynucleotide sequences or amino acid residues of a polypeptide between two sequences after aligning both sequences to maximize matching (best match) in a specific comparison region. Sequence identity is a value measured by optimally aligning and comparing two sequences in a specific comparison region, and a part of the sequence in the comparison region may be added or deleted compared to a reference sequence. Percentage sequence identity may be calculated by, for example, comparing two optimally aligned sequences in the comparison region, determining a number of positions at which identical amino acids or nucleotides occur in both sequences to obtain a number of matched positions, dividing the number of matched positions by a total number of positions within the comparison range (i.e., range size), and multiplying 100 to the result. The percentage of sequence identity may be determined by using a known sequence comparison program, examples of which include

BLASTn™ (NCBI), BLASTp™ (NCBI), CLC Main Workbench (CLC bio), MegAlign™ (DNASTAR Inc), and the like.

The term “genetic modification”, used herein, refers to an artificial alteration of a composition or structure of a genetic material of a cell.

In one aspect is provided a recombinant microorganism including a genetic modification that increases the expression of an iron storage protein (ISP) having a heme structure. An iron storage protein may be a protein that provides an intracellular iron reserve for use when external supplies are limited. The iron storage protein may be an iron storage protein derived from bacteria. Iron may be stored in the iron storage protein. The iron storage protein may be a bacterioferritin (Bfr) type.

Bfr may be an oligo-protein including an iron center and heme. The heme may be cytochrome, for example, cytochrome type b. Bfr may be protoheme IX-containing heme ferritin. E. coli-derived Bfr (EcBfr) may be composed of 24 identical subunits and include 12 heme groups at interfaces between the subunits. In Bfr, the heme may be involved in iron reduction and excretion. The Bfr may be derived from bacteria. The bacteria may belong to the genus Escherichia, Cupriavidus, Azotobacter, Brucella, Clostridium, Magnetospirillum, Mycobacterium, Neisseria, Pseudomonas, Rhodobacter, Rhodospirillum, Synechocystis, Desulfovibrio or Vibrio. The bacteria may be, for example, E. coli, C. necator, A. chroococcum, A. vinelandii, B. melitensis, C. acetobutylicum, M. magnetotacticum, M. avium, M. leprae, M. paratuberculosis, M. tuberculosis, N. gonorrhoeae, N. meningitides, N. winogradskii, P. aeruginosa, P. putida, R. capsulatus, R. sphaeroides, R. rubrum, Synechocystis sp. PCC6803, D. desulfuricans, or V. cholerae.

The genetic modification may be a genetic modification that increases a copy number of a gene encoding the iron storage protein, for example, bacterioferritin.

The recombinant microorganism may belong to the genus Escherichia, Cupriavidus, Azotobacter, Brucella, Clostridium, Magnetospirillum, Mycobacterium, Neisseria, Pseudomonas, Rhodobacter, Rhodospirillum, Synechocystis, Desulfovibrio or Vibrio. The recombinant microorganism may be, for example, E. coli, C. necator, A. chroococcum, A. vinelandii, B. melitensis, C. acetobutylicum, M. magnetotacticum, M. avium, M. leprae, M. paratuberculosis, M. tuberculosis, N. gonorrhoeae, N. meningitides, N. winogradskii, P. aeruginosa, P. putida, R. capsulatus, R. sphaeroides, R. rubrum, Synechocystis sp. PCC6803, D. desulfuricans, or V. cholerae.

The recombinant microorganism before being genetically engineered may already include a genetic modification that increases the activity of reducing nitrogen oxide to nitrogen. The genetic modification may be a genetic modification that increases a native activity of reducing nitrogen oxide to nitrogen, or a genetic modification that introduces an exogenous activity reducing nitrogen oxide into nitrogen. The genetic modification may be a genetic modification that increases the expression of a gene encoding an enzyme or protein that catalyzes or increases one or more reactions in the process of reducing nitrogen oxide to nitrogen. The reaction may be one or more of reducing NO to N₂O and reducing N₂O to N₂. The recombinant microorganism before being genetically engineered may include a genetic modification that increases activity of at least one of nitric oxide reductase and nitrous oxide reductase (NOR). The recombinant microorganism before being genetically engineered may include a genetic modification that introduces from outside activity of at least one of nitric oxide reductase and nitrous oxide reductase (NOR). For example, the recombinant microorganism may be one in which an exogenous gene encoding at least one of a nitric oxide reductase and a nitrous oxide reductase (NOR) is introduced and expression thereof is increased. The recombinant microorganism may be a microorganism of the genus Escherichia, for example, Escherichia coli.

The recombinant microorganism may have increased ability to reduce nitrogen oxide in a sample. The nitrogen oxide may be in a form of Fe(II)(L)-NO. Fe(II)(L)-NO represents that a chelating agent L and Fe²⁺ and NO are chelated to form a complex. L in the complex may be, for example, ethylenediamine, diethylenetriamine, triethylenetetraamine, hexamethylenetetraamine, N-(2-hydroxyethyl)ethylenediamine-triacetic acid (HEDTA), ethylenediamine-tetraacetic acid (EDTA), iminodiacetic acid, nitrilo-triacetic acid (NTA), or diethylenetriaminepentaacetic acid (DTPA). Accordingly, Fe(II)(L)-NO may be in a form in which nitrogen oxide such as N₂O, NO, N₂O₃, NO₂, N₂O₄, and N₂O₅ is modified to become soluble in an aqueous solution. Fe(II)(L)-NO may be formed by contacting an aqueous solution containing Fe(II)(L) with nitrogen oxide. The contacting may be mixing an aqueous medium with a liquid sample including the solubilized nitrogen oxide or contacting an aqueous medium with a sample including a gaseous nitrogen oxide. However, when the microorganisms reduce a nitrogen oxide concentration in a sample, the mechanism is not limited to this specific mechanism.

The iron storage protein, for example, bacterioferritin, may be exogenous or endogenous. The iron storage protein, for example, bacterioferritin may be present in the cytoplasm or in the cell membrane.

The iron storage protein, for example, bacterioferritin, may include a polypeptide selected from the group consisting of polypeptides having sequence identity with the amino acid sequence of SEQ ID NO: 1 of about 75% or more, about 80% or more, about 85% or more, about 90% or more, or about 95% or more, respectively. SEQ ID NO: 1 is the amino acid sequence of E. coli-derived Bfr.

A gene encoding the iron storage protein, for example, bacterioferritin, may have sequence identity of about 75% or more, about 80% or more, about 85% or more, about 90% or more, or about 95% or more, with the nucleotide sequence of SEQ ID NO: 2. SEQ ID NO: 2 is the nucleotide sequence encoding the amino acid sequence of E. coli-derived Bfr.

In the microorganism, the genetic modification may increase the expression of a gene encoding an iron storage protein, for example, bacterioferritin. The genetic modification may increase a copy number of the gene encoding an iron storage protein. The genetic modification may increase a copy number of a gene encoding a polypeptide having sequence identity of about 80% or more, about 85% or more, about 90% or more, or about 95% or more, with the amino acid sequence of SEQ ID NO: 1. The gene may have sequence identity of about 80% or more, about 85% or more, about 90% or more, or about 95% or more, with the nucleotide sequence of SEQ ID NO: 2. The genetic modification may include introducing a gene encoding bacterioferritin, for example, through a vehicle such as a vector. The gene encoding the iron storage protein may exist intrachromosomally or extrachromosomally. The introduced gene encoding an iron storage protein, for example, bacterioferritin, may be plural in number, for example, 2 or more, 5 or more, 10 or more, 50 or more, 100 or more, or 1,000 or more. The gene may be expressed constitutively or under inducible conditions by being operably linked to a constitutive promoter or an inducible promoter.

The recombinant microorganism may reduce a nitrogen oxide concentration in a sample. The reduction may include facilitating at least one of: converting nitrogen oxide to nitrous oxide (N₂O); and converting the converted nitrous oxide to nitrogen (N₂), the processes being performed by the iron storage protein, for example, bacterioferritin. The degree of facilitation may increase as the concentration of the iron storage protein increases. For example, the iron storage protein may increase expression of at least one of a nitric oxide reductase gene and a nitrous oxide reductase gene. The expression may increase as the concentration of the iron storage protein increases. In addition, the iron storage protein may increase the activity of at least one of a nitric oxide reductase gene and a nitrous oxide reductase gene. The activity may increase as the concentration of the iron storage protein increases. The nitric oxide reductase may be NorV. The nitrous oxide reductase may be NosZ. The sample may be in a liquid or gaseous state. The sample may be factory wastewater or waste gas. The sample includes any sample containing nitrogen oxide such as the above-mentioned nitrogen oxide. The nitrogen oxide may include N₂O, NO, N₂O₃, NO₂, N₂O₄, N₂O₅ or a mixture thereof.

In another aspect is provided a composition for use in reducing a nitrogen oxide concentration in a sample, the composition comprising the recombinant microorganism with a genetic modification that increases the expression of an iron storage protein (ISP) having a heme structure.

In the composition, the recombinant microorganism, sample, and nitrogen oxide are as described above. The recombinant microorganism may be in a living form, dried form, or lysed form, or in a form of an isolated iron storage protein. The dried form may be freeze-dried.

The term “reduction”, which is used in the description of the composition, refers to reducing a nitrogen oxide concentration present in a sample, and may include completely eliminating nitrogen oxide. The sample may be a gas or a liquid. The sample may not include the microorganism. The composition may further include a substance that increases solubility of the nitrogen oxide in a culture medium or a culture.

The nitrogen oxide may be in a form of Fe(II)(L)-NO, in the composition. Fe(II)(L)-NO represents that a chelating agent L and Fe²⁺ and NO are chelated to form a complex. L in the complex may be, for example, ethylenediamine, diethylenetriamine, triethylenetetraamine, hexamethylenetetraamine, N-(2-hydroxyethyl)ethylenediamine-triacetic acid (HEDTA), ethylenediamine-tetraacetic acid (EDTA), iminodiacetic acid, nitrilo-triacetic acid (NTA), or diethylenetriaminepentaacetic acid (DTPA). Accordingly, Fe(II)(L)-NO may be in a form in which nitrogen oxide such as N₂O, NO, N₂O₃, NO₂, N₂O₄, and N₂O₅ is modified to become soluble in an aqueous solution. Fe(II)(L)-NO may be formed by contacting an aqueous solution containing Fe(II)(L) with nitrogen oxide. The contacting may be mixing an aqueous medium with liquid nitrogen oxide or contacting an aqueous medium with gaseous nitrogen oxide. However, with respect to a reduction mechanism of a nitrogen oxide concentration by using the recombinant microorganism, the disclosure is not necessarily interpreted to be limited to this specific mechanism.

The composition may be used in reducing a nitrogen oxide concentration in a sample by contacting the composition with the sample. The contacting may be performed in a liquid phase. The contacting may be by, for example, contacting the sample with a culture of microorganisms cultured in a medium. The culturing may be performed under conditions in which the microorganisms proliferate. The contacting may be performed in a closed container. The contacting may include culturing or incubating the recombinant microorganisms while contacting the recombinant microorganisms with a nitrogen oxide-containing sample. The contacting includes culturing the recombinant microorganisms in a closed container under conditions in which the recombinant microorganisms proliferate.

The composition may not include other microorganisms apart from the recombinant microorganism. The composition may not include other intentionally added microorganisms apart from the recombinant microorganism. The composition may include the recombinant microorganism as a single microorganism. The recombinant microorganism may not be in a form of a microbial collection that exists in nature.

Another aspect provides a method of reducing a nitrogen oxide concentration in a sample, including reducing a nitrogen oxide concentration in a sample by contacting a nitrogen oxide-containing sample with a recombinant microorganism, wherein the recombinant microorganism includes a genetic modification that increases the expression of an iron storage protein (ISP) having a heme structure.

In the method, the recombinant microorganism, and nitrogen oxide-containing sample are as described above.

The method may not include other microorganisms apart from the recombinant microorganism. The recombinant microorganism may not include other intentionally added microorganisms. The recombinant microorganism may be composed of a single microorganism as an active microorganism. The recombinant microorganism may not be in a form of a microbial collection that exists in nature.

The nitrogen oxide may be in a form of Fe(II)(L)-NO, in the method. Fe(II)(L)-NO represents that a chelating agent L and Fe²⁺ and NO are chelated to form a complex. L in the complex may be, for example, ethylenediamine, diethylenetriamine, triethylenetetraamine, hexamethylenetetraamine, N-(2-hydroxyethyl)ethylenediamine-triacetic acid (HEDTA), ethylenediamine-tetraacetic acid (EDTA), iminodiacetic acid, nitrilo-triacetic acid (NTA), or diethylenetriaminepentaacetic acid (DTPA). Accordingly, Fe(II)(L)-NO may be in a form in which nitrogen oxide such as N₂O, NO, N₂O₃, NO₂, N₂O₄, and N₂O₅ is modified to become soluble in an aqueous solution. Fe(II)(L)-NO may be formed by contacting an aqueous solution containing Fe(II)(L) with the nitrogen oxide. The contacting may be by mixing an aqueous medium with a liquid nitrogen oxide or contacting an aqueous medium with a gaseous nitrogen oxide. However, when the microorganism reduces a nitrogen oxide concentration in a sample, the mechanism is not limited to this specific mechanism.

In the above method, the contacting may be performed in a liquid phase. The contacting may be by, for example, contacting the sample with a culture of the microorganisms in a culture medium. The culturing may be performed under conditions in which the microorganisms proliferate. The contacting may be performed in a closed container. The contacting may be performed when a growth phase of the microorganisms is at an exponential phase or at a stationary phase. The culturing may be performed under aerobic or anaerobic conditions. The contacting may be performed in a closed container under conditions in which the recombinant microorganisms may survive. The conditions in which the recombinant microorganisms may survive may be conditions in which the recombinant microorganisms are able to proliferate or are in a resting state. The contacting may be performed under conditions including an inducer. The inducer may be isopropylthio-β-galactoside (IPTG). The inducer may promote initiation of gene transcription from an inducible promoter.

In the method, the sample may be in a liquid or gaseous state. The sample may be factory wastewater or waste gas. The sample may not only be passively contacted with a culture of the recombinant microorganisms, but also may be actively contacted. The sample may be, for example, sparged in a culture medium of the recombinant microorganisms. That is, the sample may be blown through a medium or a culture medium. The sparging may be blowing from the bottom to the top of the medium or culture medium. The sparging may be injecting droplets of the sample. The nitrogen oxide may be in a form of Fe(II)(L)-NO.

In the method, the contacting may be performed batch-wise or continuously. The contacting is, for example, contacting the sample obtained in the reducing process with fresh recombinant microorganisms containing a genetic modification that increases the expression of an iron storage protein (ISP) having a heme structure. Such contacting with fresh microorganisms may be performed 2 times or more, for example, 2 times, 3 times, 5 times, or 10 times. The contacting may be continued or repeated for a period of time until a desired reduced concentration of nitrogen oxide in the sample is achieved.

Another aspect provides a method of producing the recombinant microorganism including introducing a genetic modification that increases the expression of an iron storage protein into the microorganism. The method may include preparing the recombinant microorganism, including introducing a gene encoding an iron storage protein, for example, bacterioferritin, into the microorganism. The introduction of a gene encoding an iron storage protein, for example, bacterioferritin, may include introducing a vehicle containing the gene into the microorganism. In the method, the genetic modification may include amplifying the gene, engineering a regulatory sequence of the gene, or engineering the sequence of the gene itself. The engineering may be insertion, substitution, conversion, or addition of nucleotides. The microorganism before being genetically engineered may be a recombinant microorganism including a genetic modification that increases activity of at least one of a nitric oxide reductase and a nitrous oxide reductase. The recombinant microorganism may be a microorganism of the genus Escherichia, for example, Escherichia coli.

A recombinant microorganism according to an aspect may be used to remove nitrogen oxide from a sample including nitrogen oxide.

A composition according to another aspect may be used to reduce a nitrogen oxide concentration in a sample including nitrogen oxide.

According to a method of reducing nitrogen oxide concentration in a sample including nitrogen oxide according to another aspect, a nitrogen oxide concentration in a sample may be effectively reduced.

### DETAILED DESCRIPTION

Reference will now be made in detail to embodiments, embodiments of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of at least one of the associated listed items. Expressions such as “at least one of,” when preceding a list of elements, modify the entire list of elements and do not modify the individual elements of the list.

Hereinafter, the present disclosure will be described in more detail through examples. However, these examples are intended to illustrate the present disclosure, and the scope of the present disclosure is not limited to these examples.

### Example 1: Confirmation of E. Coli Overexpressing Iron Storage Protein Gene and its Ability to Remove Fe(II)EDTA-NO

In this example, a recombinant microorganism overexpressing an iron storage protein gene was prepared by introducing an iron storage protein gene that is endogenous to the microorganism. Next, the increased ability of the resulting recombinant microorganism to reduce nitrogen in a form of Fe(II)EDTA-NO to N₂O was confirmed. E. coli was used as the recombinant microorganism.

Production of Recombinant E. coli Overexpressing Iron Storage Protein Gene

In E. coli, iron storage proteins include ferritin, putative ferritin-like protein, bacterioferritin (Bfr), and DNA-binding proteins from starved cells. The genes encoding these proteins are ftnA, ftnB, bfr, and dps, respectively.

Recombinant strains in which E. coli-derived ftnA, ftnB, bfr, and dps genes were respectively overexpressed were prepared with E. coli W3110. Specifically, E. coli W3110 was cultured in a medium, E. coli genomic DNA (gDNA) isolated from the culture was extracted, and E. coli ftnA, ftnB, bfr, and dps genes were each amplified through PCR, using the gDNA as a template, using oligonucleotides of SEQ ID NOS: 3 and 4, SEQ ID NOS: 5 and 6, SEQ ID NOS: 7 and 8, and SEQ ID NOS: 9 and 10 as primer sets, respectively. The bfr gene has the nucleotide sequence of SEQ ID NO: 2. Vector fragments were obtained by PCR amplification by using a vector pIND4 (AC Ind et al. Appl Environ Microbiol. 2009 October; 75(20): 6613-5) as a template and oligonucleotides of SEQ ID NOS: 11 and 12 as a primer set. Each of the ftnA, ftnB, bfr, and dps genes was ligated to the vector fragments by using an InFusion Cloning Kit (Clontech Laboratories, Inc.) according to the manufacture's instructions to produce vectors pIND4-ftnA, pIND4-ftnB, pIND4-bfr, and pIND4-dps overexpressing the ftnA, ftnB, bfr, and dps genes, respectively. In this regard, the gene was operably linked to an inducible promoter, that is, a lac promoter, in order that the expression would be induced by isopropylthio-β-galactoside (IPTG).

The vector overexpressing an iron storage protein and pIND4 (an empty vector) as a control group were introduced into E. coli W3110 cells by the electroporation method (Sambrook, J & Russell, D. W., New York: Cold Spring Harbor Laboratory Press, 2001), to produce W3110/pIND4, W3110/pIND4-ftnA, W3110/pIND4-ftnB, W3110/pIND4-bfr, and W3110/pIND4-dps strains. The transformed strains were obtained by selection on LB plate medium containing kanamycin (50 μg/ml).

1.2. Confirmation of Ability to Remove Fe(II)EDTA-NO by Reduction

Recombinant E. coli strains into which an iron storage protein gene was introduced, that is, W3110/pIND4, W3110/pIND4-ftnA, W3110/pIND4-ftnB, W3110/pIND4-bfr, and W3110/pIND4-dps strains, were cultured in LB medium at 30° C. and 140 rpm with shaking, and expression of iron storage protein gene was induced by adding 0.1 mM of IPTG. Next, E. coli cells in which the iron storage protein gene was overexpressed were isolated. The isolated cells were added to M9 medium containing 5 g/L of glucose and 5 mM of pH 7.0 Fe(II)EDTA-15NO to OD₆₀₀=1, to obtain a reaction mixture.

30 mL of the reaction mixture was added to a 60 mL serum bottle and incubated at 30° C. with shaking at 140 rpm. The serum bottle was maintained in an anaerobic chamber to provide anaerobic conditions. The same procedures were performed for the control group except that a control strain, that is, E. coli including an empty vector, was used. Analyses were performed after 2 hours and 4 hours of incubation.

Next, the reaction solution of the reaction serum bottle was sampled and centrifuged at 13,000 rpm to remove cells and the supernatant was separated. For the obtained supernatant, an absorbance at 420 nm (A₄₂₀) was measured by using a spectrophotometer to measure an remaining amount of substrate Fe(II)EDTA-NO.

The Fe(II)EDTA-NO complex substrate in the reaction solution was dark brown in color. Accordingly, as the Fe(II)EDTA-NO complex substrate is reduced to N₂O, the reaction solution gradually becomes lighter in color from brown, to yellow, and white, as the concentration of Fe(II)EDTA-NO decreases. Therefore, the decrease in Fe(II)EDTA-NO concentration may be confirmed by visually observing the color change of the reaction solution over time.

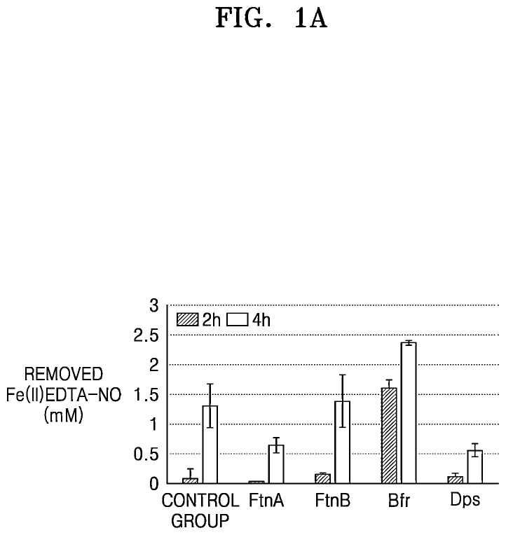

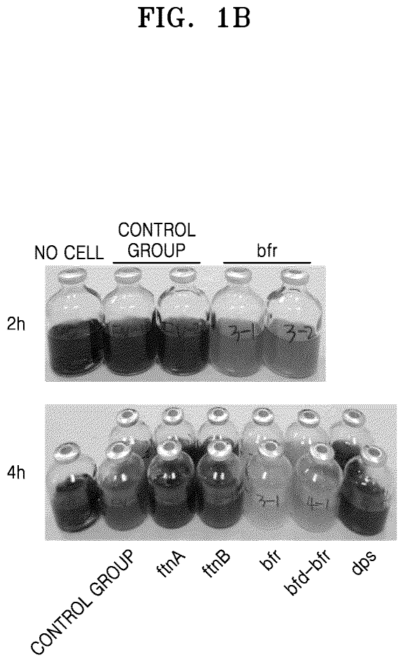

The results are shown in FIGS. 1A and 1B. FIG. 1A is a diagram showing amounts of Fe(II)EDTA-NO removed during Fe(II)EDTA-NO reduction reaction by using recombinant E. coli in which ftnA, ftnB, bfr, and dps genes are overexpressed.

FIG. 1B is a diagram comparing colors of reaction solutions of Fe(II)EDTA-NO reduction that uses recombinant E. coli in which ftnA, ftnB, bfr, and dps genes are overexpressed;

In FIGS. 1A and 1B, 2h and 4h represent results of culturing for 2 hours and 4 hours, respectively.

As shown in FIGS. 1A and 1B, the amount of Fe(II)EDTA-NO removed from the samples using E. coli W3110/pIND4-bfr strain, into which the bfr gene was introduced, significantly increased compared to the control group. This indicates that the ability of the W3110/pIND4-bfr strain to reduce Fe(II)EDTA-NO to N₂O was significantly increased.

### Example 2: Confirmation of E. coli from which Bfr Gene was Deleted and its Ability to Remove Fe(II)EDTA-NO

2.1. Construction of Recombinant E. coli with Bfr Gene Deletion

bfr, bfd and bfd-bfr genes were respectively deleted from E. coli W3110 by using the one-step inactivation method (KA Datsenko and BL Wanner, Proc Natl Acad Sci USA. 2000 Jun. 6; 97(12): 6640-5). bfd is a gene encoding bacterioferritin-associated ferredoxin.

To delete the genes respectively, PCR was performed using pKD3 vectors as a template and oligonucleotides of SEQ ID NOS: 13 and 14, SEQ ID NOS: 15 and 16, and SEQ ID NOS: 15 and 14 as primer sets. The obtained DNA fragments were electroporated into competent cells of the W3110 strain, in which lambda-red recombinases were expressed, to prepare mutant strains in which the genes were respectively deleted. Colony PCR was performed with the primer sets of SEQ ID NOS: 17 and 18, SEQ ID NOS: 19 and 20, and SEQ ID NOS: 19 and 18 to confirm respective deletion of the genes. As a result, W3110 Abfr, W3110 Abfd, and W3110 Abfd-bfr strains in which the genes were respectively deleted were obtained.

2.2. Confirmation of Reduced Ability to Remove Fe(II)EDTA-NO by Reduction

The recombinant E. coli strains, that is, W3110 Δbfr, W3110 Δbfd, and W3110 Δbfd-bfr strains, in which an iron storage protein gene was deleted, were cultured in LB medium at 30° C. with shaking at 230 rpm. Next, E. coli cells in which the iron storage protein gene was deleted were isolated. The isolated cells were added to M9 medium containing 5 g/L of glucose and 5 mM of pH 7.0 Fe(II)EDTA-15NO to OD₆₀₀=1, to obtain a reaction mixture.

30 mL of the reaction mixture was added to a 60 mL serum bottle and incubated at 30° C. with shaking at 140 rpm. The serum bottle was maintained in an anaerobic chamber to produce anaerobic conditions. The same procedures were performed for the control group except that wild-type E. coli was used. Analyses were performed after designated times, for example, 2 hours and 4 hours, after the incubation.

Next, the reaction solution of the reaction serum bottle was sampled and centrifuged at 13,000 rpm to remove cells and the supernatant was separated. For the obtained supernatant, the absorbance at 420 nm (A₄₂₀) was measured by using a spectrophotometer to measure the remaining amount of the substrate Fe(II)EDTA-NO.

The Fe(II)EDTA-NO complex substrate in the reaction solution is dark brown in color. Accordingly, as the Fe(II)EDTA-NO complex substrate is reduced to N₂O, the reaction solution gradually becomes lighter in color from brown, to yellow, and white, as the concentration of Fe(II)EDTA-NO decreases. Therefore, the decrease in Fe(II)EDTA-NO concentration may be confirmed by visually observing the color change of the reaction solution over time.

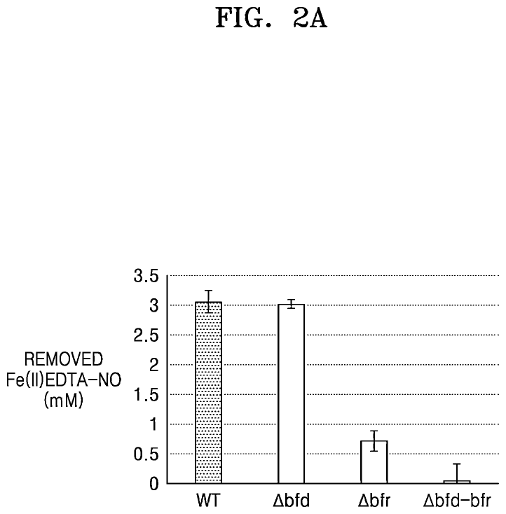

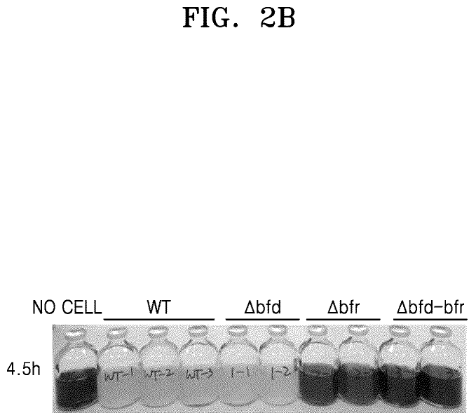

The results are shown in FIGS. 2A and 2B. FIG. 2A is a diagram showing amounts of Fe(II)EDTA-NO removed during Fe(II)EDTA-NO reduction reaction by using recombinant E. coli in which bfd, bfr, and bfd-bfr genes are respectively deleted.

FIG. 2B is a diagram comparing colors of reaction solutions of Fe(II)EDTA-NO reduction that uses recombinant E. coli in which bfd, bfr, and bfd-bfr genes are respectively deleted. In FIGS. 2A and 2B, the incubation time was 4.5 hours.

As shown in FIGS. 2A and 2B, the amount of Fe(II)EDTA-NO removed from the group using E. coli strain with the bfr gene was deleted, was significantly decreased compared to the control group. This indicates that the bfr gene significantly affects the Fe(II)EDTA-NO to N₂O reduction reaction.

### Example 3: Confirmation of Effect of Bacterioferritin Gene Overexpression on E. coli Nitric Oxide Reductase Gene Expression

3.1. Production of PnorV-IacZ E. coli Strain

In order to confirm an effect of iron storage protein (ISP) overexpression in E. coli on expression of a nitric oxide reductase V gene (norV), recombinant E. coli in which a promoter of a lacZ gene was replaced with a norV gene promoter (PnorV) on the chromosome was produced. This strain is hereinafter referred to as a PnorV-lacZ strain.

LacZ in the produced recombinant E. coli is expressed by the norV promoter, and through this, activity of the norV promoter may be evaluated. PnorV represents a norV promoter. The production process of recombinant E. coli including a PnorV-lacZ fusion gene on the chromosome is as follows.

To construct a norV promoter template vector, E. coli W3110 was cultured in a medium, E. coli gDNA isolated from the culture was extracted, and PCR was performed using the gDNA as a template and oligonucleotides of SEQ ID NOS: 21 and 22 as a primer set to amplify the E. coli norV gene promoter region. The amplified norV gene promoter region has a nucleotide sequence of SEQ ID NO: 23. Vector fragments were obtained by PCR amplification by using pMtrc9 vectors (JH Park et al. ACS Synth Biol. 2012 Nov. 16; 1(11): 532-40) as a template and oligonucleotides of SEQ ID NOS: 24 and 25 as a primer set. pMnorV template vectors were constructed by ligating norV gene promoters to vector fragments by using an InFusion Cloning Kit (Clontech Laboratories, Inc.) according to the manufacturer's instruction.

PCR was performed by using the pMnorV vectors as a template and the oligonucleotides of SEQ ID NOS: 26 and 27 as primers. The obtained DNA fragments were electroporated into competent cells of the W3110 strain, in which a lambda-red recombinase was expressed, to prepare mutant strains in which ISP genes were respectively deleted. Colony PCR was performed with primers of SEQ ID NOS: 28 and 29 to confirm replacement of the lacZ gene promoter. As a result, a W3110 PnorV-lacZ strain in which the lacZ gene was replaced with the norV promoter was obtained.

3.2. Production of Recombinant E. coli Overexpressing Iron Storage Protein Gene

Recombinant E. coli overexpressing an iron storage protein gene was prepared as described in “1.1” of Example 1, using the produced PnorV-lacZ strain as a host.

As a result, PnorV-lacZ/pIND4-ftnA, PnorV-lacZ/pIND4-ftnB, PnorV-lacZ/pIND4-bfr, and PnorV-lacZ/pIND4-dps strains overexpressing ftnA, ftnB, bfr, and dps genes were prepared. PnorV-lacZ/pIND4 (an empty vector) was a control strain.

3.3. Confirmation of norV Promoter Activity Under Conditions of Removing Fe(II)EDTA-NO by Reduction

The recombinant E. coli into which an iron storage protein gene was introduced, that is, PnorV-lacZ/pIND4 (empty vector), PnorV-lacZ/pIND4-ftnA, PnorV-lacZ/pIND4-ftnB, PnorV-lacZ/pIND4-bfr, and PnorV-lacZ/pIND4-dps strains were cultured in LB medium at 30° C. with shaking at 140 rpm, and 0.1 mM of IPTG was added to induce expression of the iron storage protein genes. Next, recombinant E. coli cells in which the iron storage protein gene was overexpressed were isolated. The isolated cells were added to M9 medium containing 5 g/L of glucose and 5 mM of pH 7.0 Fe(II)EDTA-NO to OD₆₀₀=1, to obtain a reaction mixture.

30 mL of the reaction mixture was added to a 60 mL serum bottle and incubated at 30° C. with shaking at 140 rpm. The serum bottle was maintained in an anaerobic chamber to make anaerobic conditions. The same procedures were performed for the control group except that the control strain, that is, E. coli including an empty vector, was used. Next, after 2 hours of culture, the cell fluid was collected to have OD of 1. The cells were disrupted by mixing the collected sample with a Lysis buffer, Bugbuster Master mix (Merck). A level of beta-galactosidase activity of the obtained cell lysate was measured by using Abcam's Beta Galactosidase Detection Kit (fluorometric) (ab176721). By measuring the beta-galactoxidase activity, the enzyme activity of LacZ, that is, a beta-galactoxidase, expressed by the replaced norV promoter may be measured. Specifically, fluorescein di-β-D-galactopyranoside (FDG) was added to the obtained cell lysate, and fluorescence was measured at Ex/Em=490/525 nm. FDG is a non-fluorescent reactive substance that is decomposed by LacZ and converted into fluorescein, a powerful fluorescent substance. 490 nm and 525 nm represent excitation and emission wavelengths of fluorescein, respectively.

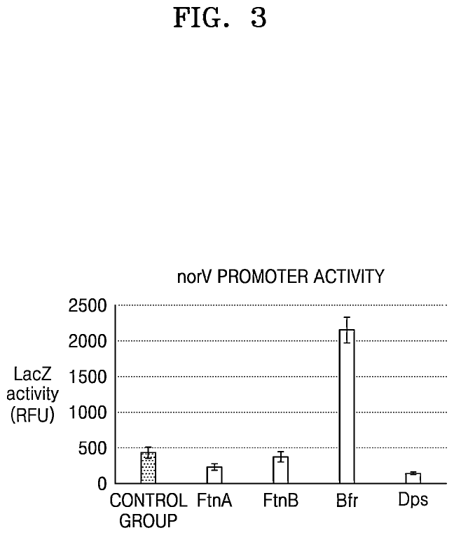

FIG. 3 is a bar graph showing results of measuring norV promoter activity of a recombinant strain overexpressing iron storage protein (ISP) gene under Fe(II)EDTA-NO reduction conditions. As shown in FIG. 3, a recombinant strain overexpressing bfr showed increased LacZ activity compared to the control strain (E. coli including an empty vector). This is due to increased norV promoter activity. On the other hand, for strains overexpressing other ISP genes, ftnA, ftnB, or dps, LacZ activity was similar to that of the control strain, or rather decreased.

These results indicate that a Bfr protein increases the expression of a nitric oxide reductase gene (norV) of E. coli, thereby increasing Fe(II)EDTA-NO removal by reduction.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A recombinant microorganism comprising a genetic modification that increases the expression of an iron storage protein (ISP) having a heme structure, wherein the microorganism belongs to the genus Escherichia, and the iron storage protein is bacterioferritin (Bfr).

2. The recombinant microorganism of claim 1, wherein the bacterioferritin comprises a polypeptide having sequence identity of 75% or more with the amino acid sequence of SEQ ID NO: 1.

3. A composition for reducing a nitrogen oxide concentration in a sample, the composition comprising a recombinant microorganism comprising a genetic modification that increases the expression of iron storage protein (ISP) having a heme structure.

4. The composition of claim 3, wherein the iron storage protein increases the expression of a nitric oxide reductase gene (norV).

5. The composition of claim 3, wherein the iron storage protein is bacterioferritin (Bfr).

6. The composition of claim 3, wherein the recombinant microorganism belongs to a genus Escherichia.

7. The composition of claim 5, wherein the bacterioferritin comprises a polypeptide having sequence identity of 75% or more with the amino acid sequence of SEQ ID NO: 1.

8. The composition of claim 3, wherein the nitrogen oxide is in a form of Fe(II)(L)-NO in which a chelating agent L and Fe²⁺ and NO are chelated to form a complex.

9. A method of reducing a nitrogen oxide concentration in a sample, comprising reducing a nitrogen oxide concentration in a sample by contacting a nitrogen oxide-containing sample with recombinant microorganisms including a genetic modification that increases the expression of an iron storage protein (ISP) having a heme structure.

10. The method of claim 9, wherein the iron storage protein increases the expression of a nitric oxide reductase (Nor) gene.

11. The method of claim 9, wherein the iron storage protein is bacterioferritin (Bfr).

12. The method of claim 9, wherein the recombinant microorganism belongs to a genus Escherichia.

13. The method of claim 11, wherein the bacterioferritin comprises a polypeptide having sequence identity of 75% or more with the amino acid sequence of SEQ ID NO: 1.

14. The method of claim 9, wherein the nitrogen oxide is in a form of Fe(II)(L)-NO in which a chelating agent L and Fe²⁺ and NO are chelated to form a complex.

15. The method of claim 9, wherein the contacting is performed in a closed container.

16. The method of claim 9, wherein the contacting comprises culturing or incubating the recombinant microorganisms while being in contact with the nitrogen oxide-containing sample.

17. The method of claim 9, wherein the contacting comprises culturing the recombinant microorganisms in a closed container under conditions in which the recombinant microorganisms proliferate.


SEQUENCE LISTING (29 sequences)

SEQ ID NO: 1 - protein, 158 aa, Escherichia coli
MKGDTKVINYLNKLLGNELVAINQYFLHARMFKNWGLKRLNDVEYHESIDEMKHADRYIERILFLEGLPNLQDLGKLNIGEDVEEMLRSDLALELDGAKNLREAIGYADSVHDYVSRDMMIEILRDEEGHIDWLETELDLIQKMGLQNYLQAQIREEG

SEQ ID NO: 2 - DNA, 477 bp, Escherichia coli
  source 1..477: mol_type = genomic DNA
atgaaaggtgatactaaagttataaattatctcaacaaactgttgggaaatgagcttgtcgcaatcaatcagtactttctccatgcccgaatgtttaaaaactggggtctcaaacgtctcaatgatgtggagtatcatgaatccattgatgagatgaaacacgccgatcgttatattgagcgcattctttttctggaaggtcttccaaacttacaggacctgggcaaactgaacattggtgaagatgttgaggaaatgctgcgttctgatctggcacttgagctggatggcgcgaagaatttgcgtgaggcaattggttatgccgatagcgttcatgattacgtcagccgcgatatgatgatagaaattttgcgtgatgaagaaggccatatcgactggctggaaacggaacttgatctgattcagaagatgggcctgcaaaattatctgcaagcacagatccgcgaagaaggttga

SEQ ID NO: 3 - DNA, 39 bp, synthetic construct
  source 1..39: mol_type = other DNA
taaagaggagaaattaaccatgctgaaaccagaaatgat

SEQ ID NO: 4 - DNA, 41 bp, synthetic construct
  source 1..41: mol_type = other DNA
gtggtggtggtggtgctcgaggttttgtgtgtcgagggtag

SEQ ID NO: 5 - DNA, 39 bp, synthetic construct
  source 1..39: mol_type = other DNA
taaagaggagaaattaaccatggcaaccgctggaatgct

SEQ ID NO: 6 - DNA, 40 bp, synthetic construct
  source 1..40: mol_type = other DNA
gtggtggtggtggtgctcgagatgcagctggtgtgacacg

SEQ ID NO: 7 - DNA, 39 bp, synthetic construct
  source 1..39: mol_type = other DNA
taaagaggagaaattaaccatgaaaggtgatactaaagt

SEQ ID NO: 8 - DNA, 41 bp, synthetic construct
  source 1..41: mol_type = other DNA
gtggtggtggtggtgctcgagaccttcttcgcggatctgtg

SEQ ID NO: 9 - DNA, 39 bp, synthetic construct
  source 1..39: mol_type = other DNA
taaagaggagaaattaaccatgagtaccgctaaattagt

SEQ ID NO: 10 - DNA, 41 bp, synthetic construct
  source 1..41: mol_type = other DNA
gtggtggtggtggtgctcgagttcgatgttagactcgataa

SEQ ID NO: 11 - DNA, 38 bp, synthetic construct
  source 1..38: mol_type = other DNA
ctcgagcaccaccaccaccaccactaaaattagctgag

SEQ ID NO: 12 - DNA, 36 bp, synthetic construct
  source 1..36: mol_type = other DNA
catggttaatttctcctctttaattctagatgtgtg

SEQ ID NO: 13 - DNA, 69 bp, synthetic construct
  source 1..69: mol_type = other DNA
aagcgaaggagtcaaaaaatgaaaggtgatactaaagttataaattatctgtgtaggctggagctgctt

SEQ ID NO: 14 - DNA, 69 bp, synthetic construct
  source 1..69: mol_type = other DNA
tctggcctggatctcttattaaccgggagggttctccctcccgacacggccatatgaatatcctcctta

SEQ ID NO: 15 - DNA, 69 bp, synthetic construct
  source 1..69: mol_type = other DNA
aaataagaactattttcatttatttttgaagaaagtatacgggagtctctgtgtaggctggagctgctt

SEQ ID NO: 16 - DNA, 69 bp, synthetic construct
  source 1..69: mol_type = other DNA
tcacttcacgcgcggcccggacacatttaccgcactgattgccgaccggacatatgaatatcctcctta

SEQ ID NO: 17 - DNA, 20 bp, synthetic construct
  source 1..20: mol_type = other DNA
gtgaagtgatggaggatgaa

SEQ ID NO: 18 - DNA, 20 bp, synthetic construct
  source 1..20: mol_type = other DNA
atgatactgctgattgcctc

SEQ ID NO: 19 - DNA, 20 bp, synthetic construct
  source 1..20: mol_type = other DNA
gtatcgccgcattttttgtc

SEQ ID NO: 20 - DNA, 20 bp, synthetic construct
  source 1..20: mol_type = other DNA
atgatactgctgattgcctc

SEQ ID NO: 21 - DNA, 40 bp, synthetic construct
  source 1..40: mol_type = other DNA
tctccctatagtgagatcttaattcgccagcacatcaacg

SEQ ID NO: 22 - DNA, 40 bp, synthetic construct
  source 1..40: mol_type = other DNA
gccaaaacagccaagcttgctagacatagcaacctcaatt

SEQ ID NO: 23 - DNA, 216 bp, Escherichia coli
  source 1..216: mol_type = genomic DNA
attcgccagcacatcaacggaaaaactcatctttgcctcactgtcaatttgactatagatattgtcatatcgaccatttgattgatagtcattttgactactcattaatgggcataattttatttatagagtaaaaacaatcagataaaaaactggcacgcaatctgcaattagcaagacatctttttagaacacgctgaataaattgaggttgct

SEQ ID NO: 24 - DNA, 29 bp, synthetic construct
  source 1..29: mol_type = other DNA
gcaagcttggctgttttggcggatgagag

SEQ ID NO: 25 - DNA, 29 bp, synthetic construct
  source 1..29: mol_type = other DNA
gatctcactatagggagaccggcagatcc

SEQ ID NO: 26 - DNA, 69 bp, synthetic construct
  source 1..69: mol_type = other DNA
tggcacgacaggtttcccgactggaaagcgggcagtgagcgcaacgcaattaggtgacactatagaacg

SEQ ID NO: 27 - DNA, 69 bp, synthetic construct
  source 1..69: mol_type = other DNA
cagtcacgacgttgtaaaacgacggccagtgaatccgtaatcatggtcatagcaacctcaatttattca

SEQ ID NO: 28 - DNA, 20 bp, synthetic construct
  source 1..20: mol_type = other DNA
caccatcaaacaggattttc

SEQ ID NO: 29 - DNA, 20 bp, synthetic construct
  source 1..20: mol_type = other DNA
aaatgtgagcgagtaacaac